(19) United States
(12) Patent Application Publication
(10) Pub. No.: US 2022/0212031 A1
(43) Pub. Date: Jul. 7, 2022

(54) DEVICES AND SYSTEMS FOR IMPLEMENTING THERAPEUTIC TREATMENTS OF LIGHT

(71) Applicant: KNOW Bio, LLC, Durham, NC (US)

(72) Inventors: F. Neal Hunter, Durham, NC (US); John A. Demos, Durham, NC (US)

(21) Appl. No.: 17/656,141

(22) Filed: Mar. 23, 2022

Related U.S. Application Data
(63) Continuation-in-part of application No. 17/162,259, filed on Jan. 29, 2021.
Application No. 17/162,259 is a continuation-in-part of application No. 17/117,889, filed on Dec. 10, 2020, now Pat. No. 11,147,984.
Application No. 17/117,889 is a continuation-in-part of application No. 17/410,154, filed on Aug. 24, 2021.
Application No. 17/410,154 is a continuation of application No. 17/117,889, filed on Dec. 10, 2020, now Pat. No. 11,147,984.
(60) Provisional application No. 63/123,631, filed on Dec. 10, 2020.
(60) Provisional application No. 63/075,010, filed on Sep. 4, 2020.
(60) Provisional application No. 63/074,970, filed on Sep. 4, 2020.
(60) Provisional application No. 63/065,357, filed on Aug. 13, 2020.
(60) Provisional application No. 62/991,903, filed on Mar. 19, 2020.

(51) Int. Cl. A61N 5/06

(52) U.S. Cl. CPC A61N 5/0624; A61N 2005/0606; A61N 2005/0632; A61N 2005/0644; A61N 2005/0662

(57) ABSTRACT

Devices and systems for impinging light on tissue to induce one or more biological effects and, more particularly, illumination devices and related systems for implementing therapeutic treatments of light are disclosed. Systems may include illumination devices that are configured to provide phototherapy for a variety of medical indications and/or health-related benefits. Illumination devices may be connected to systems that administer and/or monitor multiple illumination devices across multiple geographic regions to compile regional and/or global information related to phototherapeutic usage. Certain aspects relate to system elements, such as local devices and/or servers that are capable of generating treatment protocols for illumination devices based on diagnostic information. After treatment protocols are implemented by illumination devices, administered treatment information along with location information may be provided to the local devices and/or servers.

### RELATED APPLICATIONS

This application is a continuation-in-part of U.S. patent application Ser. No. 17/162,259, filed Jan. 29, 2021, which is a continuation-in-part of U.S. patent application Ser. No. 17/117,889, filed Dec. 10, 2020, now U.S. Pat. No. 11,147,984.

This application is further a continuation-in-part of U.S. patent application Ser. No. 17/410,154, filed Aug. 24, 2021, which is a continuation of U.S. patent application Ser. No. 17/117,889, filed Dec. 10, 2020, now U.S. Pat. No. 11,147,984.

U.S. patent application Ser. No. 17/117,889 claims the benefit of: provisional patent application Ser. No. 63/123,631, filed Dec. 10, 2020; provisional patent application Ser. No. 63/075,010, filed Sep. 4, 2020; provisional patent application Ser. No. 63/074,970, filed Sep. 4, 2020; provisional patent application Ser. No. 63/065,357, filed Aug. 13, 2020; and provisional patent application Ser. No. 62/991,903, filed Mar. 19, 2020,

The disclosures of the above-referenced applications are hereby incorporated herein by reference in their entireties.

### FIELD OF THE DISCLOSURE

The present disclosure relates generally to devices and systems for impinging light on tissue to induce one or more biological effects and, more particularly, to illumination devices and related systems for implementing therapeutic treatments of light.

### BACKGROUND

Phototherapy, or light therapy, involves exposure of the body to light to induce biological effects and promote various health-related medical benefits. Advancements in therapeutic light treatments have demonstrated beneficial results for inactivating and/or reducing viral loads of infectious diseases. Phototherapeutic light treatments have also demonstrated other health-related benefits, including the promotion of hair growth, treatment of skin or tissue inflammation such as acne, promoting tissue or skin healing or rejuvenation, enhancing wound healing, pain management, reduction of wrinkles, scars, stretch marks, varicose veins, and spider veins, treating cardiovascular disease, treating erectile dysfunction, treating microbial infections, treating hyperbilirubinemia, and treating various oncological and non-oncological diseases and disorders including diseases induced by human papillomavirus (HPV).

Various mechanisms by which phototherapy has been suggested to provide therapeutic benefits include inactivating and inhibiting growth of microorganisms and pathogens, increasing circulation (e.g., by increasing the formation of new capillaries), stimulating the production of collagen, stimulating the release of adenosine triphosphate (ATP), enhancing porphyrin production, reducing excitability of nervous system tissues, modulating fibroblast activity, increasing phagocytosis, inducing thermal effects, stimulating tissue granulation and connective tissue phagocytosis, reducing inflammation, and stimulating acetylcholine release. Phototherapy has also been suggested to stimulate cells to produce nitric oxide, which may act as a signaling messenger, cytotoxin, antiapoptotic agent, antioxidant, and regulator of microcirculation. Nitric oxide is recognized to relax vasculature smooth muscles, dilate blood vessels, inhibit aggregation of platelets, and modulate T-cell mediated immune response. Generally, phototherapy shows promise for improving health and/or treating myriad medical conditions.

The art continues to seek improved phototherapeutic light treatments providing desirable health-related benefits while being capable of overcoming challenges associated with conventional phototherapeutic light treatments.

### SUMMARY

The present disclosure relates generally to devices and systems for impinging light on tissue to induce one or more biological effects and, more particularly, to illumination devices and related systems for implementing therapeutic treatments of light. Systems may include illumination devices that are configured to provide phototherapy for a variety of medical indications and/or health-related benefits. Illumination devices may be connected to systems that administer and/or monitor multiple illumination devices across multiple geographic regions to compile regional and/or global information related to phototherapeutic usage. Certain aspects relate to system elements, such as local devices and/or servers that are capable of generating treatment protocols for illumination devices based on diagnostic information. After treatment protocols are implemented by illumination devices, administered treatment information along with location information may be provided to the local devices and/or servers.

In one aspect, an illumination device for phototherapeutic delivery of light comprises; a light source; a communication interface; and a control system associated with the communication interface, the control system configured to a collect diagnostic information, implement a treatment protocol, and send the diagnostic information and administered light treatment information associated with implementing the treatment protocol to a server via the communication interface. The illumination device may further comprise one or more of a sensor and a camera associated with the control system, the one or more of the sensor and the camera being configured to collect at least a portion of the diagnostic information. In certain embodiments, the control system is configured with a pre-configured treatment protocol, and the treatment protocol is modified from the pre-configured treatment protocol based on the diagnostic information. In certain embodiments, the control system is configured to determine the treatment protocol based on the diagnostic information. In certain embodiments, the control system is further configured to receive the treatment protocol from the server based on the diagnostic information. In certain embodiments, the control system is further configured to determine location information associated with the administered light treatment information and send the location information to the server.

In another aspect, an illumination device for phototherapeutic delivery of light, the illumination device comprises; a light source; a communication interface; and a control system associated with the communication interface, the control system configured to implement a treatment protocol, determine location information associated with the treatment protocol, and send the location information to a server via the communication interface. In certain embodiments, the location information comprises a global positioning system (GPS) location. In certain embodiments, the control system is further configured to: collect diagnostic information; send the diagnostic information to the server; and receive the treatment protocol from the server and control the light source to implement the treatment protocol. In certain embodiments, the control system is further configured to send the diagnostic information to a local device before sending the diagnostic information to the server. In certain embodiments, the control system is further configured to determine the location information before sending the diagnostic information to the server. In certain embodiments, the control system is further configured to determine the location information after receiving the treatment protocol from the server. In certain embodiments, at least one of the location information, the diagnostic information, and the treatment protocol comprises encrypted data. In certain embodiments, the treatment protocol comprises a pre-configured treatment protocol that is associated with the control system. In certain embodiments, the control system is further configured to receive the treatment protocol from a local device that is in communication with the control system and the server. In certain embodiments, the control system is further configured to send administered light treatment information to the server, the administered light treatment information comprising one or more of a wavelength of light and a dose of light associated with administered light treatment.

In another aspect, a system for phototherapeutic delivery of light comprises; a server; and a server-side application associated with the server, the server-side application configured to: receive diagnostic information from at least one of an illumination device and a local device that is in communication with the illumination device; generate a treatment protocol based on the diagnostic information; send the treatment protocol to the illumination device; and receive location information associated with administered light treatment information after the treatment protocol is implemented by the illumination device. In certain embodiments, the server-side application is configured to compile geospatial information based on: a plurality of treatment protocols generated for a plurality of illumination devices; and location information associated with administered light treatment information received from the plurality of illumination devices. In certain embodiments, the server-side application is further configured to receive additional user information together with the diagnostic information, the additional user information comprising one or more of a medical history and demographics of a user. In certain embodiments, the server-side application is configured to associate additional user information with the diagnostic information, the additional user information comprising one or more of a medical history and demographics of a user. In certain embodiments, one or more of the diagnostic information, the treatment protocol, and the location information comprises encrypted data. In certain embodiments, the server comprises an artificial intelligence library that is used to generate the treatment protocol based on the diagnostic information. In certain embodiments, the administered light treatment information comprises one or more of a wavelength of light and a dose of light implemented by the illumination device.

In another aspect, a system for phototherapeutic delivery of light comprises; a server; and a server-side application associated with the server, the server-side application configured to: receive administered light treatment information from a plurality of illumination devices, the administered light treatment information being associated with location information; and provide data for compiling geospatial information based on the administered light treatment information and the location information. In certain embodiments, the administered light treatment information comprises one or more of a wavelength of light and a dose of light associated with administered light treatments implemented by the plurality of illumination devices. In certain embodiments, the administered light treatment information is associated with one or more of a user's diagnostic information, medical history, and demographics. In certain embodiments, the server-side application is further configured to receive the administered light treatment information from a local device that is in communication with the plurality of illumination devices. In certain embodiments, the administered light treatment information comprises encrypted data.

In another aspect, any of the foregoing aspects individually or together, and/or various separate aspects and features as described herein, may be combined for additional advantage. Any of the various features and elements as disclosed herein may be combined with one or more other disclosed features and elements unless indicated to the contrary herein.

Those skilled in the art will appreciate the scope of the present disclosure and realize additional aspects thereof after reading the following detailed description of the preferred embodiments in association with the accompanying drawing figures.

### BRIEF DESCRIPTION OF THE DRAWING FIGURES

The accompanying drawing figures incorporated in and forming a part of this specification illustrate several aspects of the disclosure, and together with the description serve to explain the principles of the disclosure.

FIG. 1 is a schematic view of a system for administering and monitoring phototherapy treatments of multiple illumination devices at various geographic locations.

FIG. 2 is a schematic view of the system of FIG. 1 with further details provided for one of the illumination devices.

FIG. 3 is a call-flow diagram illustrating an implementation of the system of FIGS. 1 and 2 where a server determines a treatment protocol for the illumination device based on received diagnostic and/or user information from the illumination device and/or a local device.

FIG. 4 is a call-flow diagram illustrating another implementation of the system of FIGS. 1 and 2 where the local device determines a treatment protocol for the illumination device and implemented treatment and location information is sent to the server.

FIG. 5 is a call-flow diagram illustrating another implementation of the system of FIGS. 1 and 2 where the local device collects diagnostic information and associates the diagnostic information with a user ID independently from the illumination device.

FIG. 6 is a call-flow diagram illustrating another implementation of the system of FIGS. 1 and 2 where the illumination device is pre-configured with one or more treatment protocols that may be implemented.

FIG. 7A is a perspective view of an exemplary illumination device that is configured to direct light emissions within or through a body cavity, such as an oral cavity.

FIG. 7B is a side view of the illumination device of FIG. 7A.

FIG. 8A is an exploded view of an illumination device embodied as a wearable cap for delivering phototherapy to a scalp and/or brain of a user.

FIG. 8B is a bottom plan view of a flexible printed circuit board (FPCB) from the illumination device of FIG. 8A illustrating light emitters and standoffs arranged thereon.

FIG. 9 is an illustration representing a continuous glucose monitor (CGM) with an incorporated light source capable of delivering foreign body response (FBR)-modulating light to a host's skin during monitoring.

FIG. 10 is an illustration representing a CGM that is similar to the CGM of FIG. 9 and further includes a corresponding light delivery structure capable of delivering FBR-modulating light beneath the host's skin during monitoring.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element such as a layer, region, or substrate is referred to as being “on” or extending “onto” another element, it can be directly on or extend directly onto the other element or intervening elements may also be present. In contrast, when an element is referred to as being “directly on” or extending “directly onto” another element, there are no intervening elements present. Likewise, it will be understood that when an element such as a layer, region, or substrate is referred to as being “over” or extending “over” another element, it can be directly over or extend directly over the other element or intervening elements may also be present. In contrast, when an element is referred to as being “directly over” or extending “directly over” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Relative terms such as “below” or “above” or “upper” or “lower” or “horizontal” or “vertical” may be used herein to describe a relationship of one element, layer, or region to another element, layer, or region as illustrated in the Figures. It will be understood that these terms and those discussed above are intended to encompass different orientations of the device in addition to the orientation depicted in the Figures.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Embodiments are described herein with reference to schematic illustrations of embodiments of the disclosure. As such, the actual dimensions of the layers and elements can be different, and variations from the shapes of the illustrations as a result, for example, of manufacturing techniques and/or tolerances, are expected. For example, a region illustrated or described as square or rectangular can have rounded or curved features, and regions shown as straight lines may have some irregularity. Thus, the regions illustrated in the figures are schematic and their shapes are not intended to illustrate the precise shape of a region of a device and are not intended to limit the scope of the disclosure. Additionally, sizes of structures or regions may be exaggerated relative to other structures or regions for illustrative purposes and, thus, are provided to illustrate the general structures of the present subject matter and may or may not be drawn to scale. Common elements between figures may be shown herein with common element numbers and may not be subsequently re-described.

The present disclosure relates generally to devices and systems for impinging light on tissue to induce one or more biological effects and, more particularly, to illumination devices and related systems for implementing therapeutic treatments of light. Systems may include illumination devices that are configured to provide phototherapy for a variety of medical indications and/or health-related benefits. Illumination devices may be connected to systems that administer and/or monitor multiple illumination devices across multiple geographic regions to compile regional and/or global information related to phototherapeutic usage. Certain aspects relate to system elements, such as local devices and/or servers that are capable of generating treatment protocols for illumination devices based on diagnostic information. After treatment protocols are implemented by illumination devices, administered light treatment information along with location information may be provided to the local devices and/or servers.

Light, or phototherapeutic light, may be administered at one or more wavelengths with one or more corresponding doses to induce one or more biological effects for recipient tissue. Biological effects may include at least one of inactivating and inhibiting growth of one or more combinations of microorganisms and pathogens, including but not limited to viruses, bacteria, fungi, and other microbes, among others. Biological effects may also include one or more of upregulating and/or downregulating a local immune response, stimulating enzymatic generation of nitric oxide to increase endogenous stores of nitric oxide, releasing nitric oxide from endogenous stores of nitric oxide, inducing an anti-inflammatory effect, promoting increased blood flow in the brain for the treatment of dementia, promotion of hair growth, and/or modulation of foreign body responses (FBR) in tissues. In certain aspects, light may be referred to as nitric oxide modulating light to increase concentrations of unbound nitric oxide within living tissue. Light may also be administered at one or more wavelengths as a pre-exposure prophylaxis or a post-exposure prophylaxis in order to eliminate pathogens in or on tissue of the upper respiratory tract and/or amplify host defense systems. Embodiments of the present disclosure may be used to prevent and/or treat respiratory infections and other infectious diseases.

Wavelengths of light may be selected based on at least one intended biological effect for one or more of the targeted tissues and the targeted microorganisms and/or pathogens. In certain aspects, wavelengths of light may include visible light in any number of wavelength ranges based on the intended biological effect. Further aspects involve light impingement on tissue for multiple microorganisms and/or multiple pathogenic biological effects, either with light of a single peak wavelength or a combination of light with more than one peak wavelength. Devices and methods for light treatments include those that provide light doses for inducing biological effects on various targeted pathogens and targeted tissues with increased efficacy and reduced cytotoxicity. Light doses may include various combinations of irradiances, wavelengths, and exposure times, and such light doses may be administered continuously or discontinuously with a number of pulsed exposures.

Certain aspects of the present disclosure generally relate to devices and related systems for promoting various health-related benefits, such as treating, preventing, and/or reducing the biological activity of pathogens while they are in one or more areas of the body. In certain aspects, related devices and methods may prevent or reduce infections by reducing microbial load, decreasing the ability for penetration into cells at the site of infection, and amplifying host defense systems, all of which may minimize or avoid the need for traditional antimicrobial medicines. In further aspects, related devices and methods for light irradiation of tissues may be provided to supplement and/or enhance the effects of traditional antimicrobial medicines.

The term “phototherapy” relates to the therapeutic use of light. As used herein, phototherapy may be used to promote various health-related benefits. The mechanisms by which certain wavelengths of light are effective can vary, depending on the wavelength that is administered and the targeted biological effect. Biological effects, including antimicrobial effects, can be induced over a wide range of wavelengths, including ultraviolet (UV) ranges, visible light ranges, and infrared (IR) ranges, and combinations thereof.

Various wavelengths of visible light may be irradiated on human tissue with little or no impact on tissue viability. In certain embodiments, various wavelengths of visible light may elicit antimicrobial and/or anti-pathogenic behavior in corresponding tissues, including any of the aforementioned biological effects. For example, light with a peak wavelength in a range from 400 nanometers (nm) to 450 nm may inactivate microorganisms that are in a cell-free environment and/or inhibit replication of microorganisms that are in a cell-associated environment and/or stimulate enzymatic generation of nitric oxide, while also upregulating a local immune response in target tissue. In this regard, light with a peak wavelength in a range from 400 nm to 450 nm may be well suited for fighting invading viral and/or bacterial pathogens and corresponding diseases that may originate in the respiratory tract, including Orthomyxoviridae (e.g., influenza), common colds, coronavirida (e.g., coronavirus), picornavirus infections, tuberculosis, pneumonia, bronchitis, and sinusitis. In certain embodiments, red or near-infrared (NIR) light (e.g., peak wavelength range from 630 nm to 1,000 nm) may be useful to provide anti-inflammatory effects and/or to promote vasodilation. Anti-inflammatory effects may be useful in treating disorders, particularly microbial disorders that result in inflammation along the respiratory tract. In this regard, red light may be used as part of treatment protocols that reduce any tissue inflammation that may result from exposure to blue light, which may positively impact cell viability, thereby lowering cytotoxicity even further. A decrease in inflammation can be beneficial when treating viral infections, particularly when a virus can elicit a cytokine storm and/or inflammation can result in secondary bacterial infections. Accordingly, the combination of blue light, such as light at around 425 nm, and red light at one or more anti-inflammatory wavelengths, can provide a desirable combination of biological effects.

Depending on the application, other wavelength ranges of light may also be administered to human tissue. For example, UV light (e.g., UV-A light having a peak wavelength in a range of from 315 nm to 400 nm, UV-B light having a peak wavelength in a range of from 280 nm to 315 nm, and UV-C light having a peak wavelength in a range from 200 nm to 280 nm) may be effective for inactivating microorganisms that are in a cell-free environment, inhibiting replication of microorganisms that are in a cell-associated environment, and/or stimulating enzymatic generation of nitric oxide. However, overexposure to UV light may lead to cytotoxicity concerns in associated tissue. It may therefore be desirable to use shorter cycles and/or lower doses of UV light than corresponding treatments with only visible light. In certain embodiments, light with a peak wavelength in a range from 385 nm to 450 nm may be provided to elicit an antimicrobial and/or anti-pathogenic effect. In further embodiments, such wavelengths of light may be used in treatment protocols that also administer anti-inflammatory light.

Doses of light to induce one or more biological effects may be administered with one or more light characteristics, including peak wavelengths, radiant flux, and irradiance to target tissues. Irradiances to target tissues may be provided in a range from 0.1 milliwatts per square centimeter (mW/cm²) to 200 mW/cm², or in a range from 5 mW/cm² to 200 mW/cm², or in a range from 5 mW/cm² to 100 mW/cm², or in a range from 5 mW/cm² to 60 mW/cm², or in a range from 60 mW/cm² to 100 mW/cm², or in a range from 100 mW/cm² to 200 mW/cm². Such irradiance ranges may be administered in one or more of continuous wave and pulsed configurations, including light-emitting diode (LED)-based photonic devices that are configured with suitable power (radiant flux) to irradiate a target tissue with any of the above-described ranges. A light source for providing such irradiance ranges may be configured to provide radiant flux values from the light source of at least 5 mW, or at least 10 mW, or at least 15 mW, or at least 20 mW, or at least 30 mW, or at least 40 mW, or at least 50 mW, or at least 100 mW, or at least 200 mW, or in a range of from 5 mW to 200 mW, or in a range of from 5 mW to 100 mW, or in a range of from 5 mW to 60 mW, or in a range of from 5 mW to 30 mW, or in a range of from 5 mW to 20 mW, or in a range of from 5 mW to 10 mW, or in a range of from 10 mW to 60 mW, or in a range of from 20 mW to 60 mW, or in a range of from 30 mW to 60 mW, or in a range of from 40 mW to 60 mW, or in a range of from 60 mW to 100 mW, or in a range of from 100 mW to 200 mW, or in a range of from 200 mW to 500 mW, or in another range specified herein. Depending on the configuration of one or more light sources, the corresponding illumination device, and the distance away from a target tissue, the radiant flux value for the light source may be higher than the irradiance value at the tissue.

While certain peak wavelengths for certain target tissue types may be administered with irradiances up to 1 W/cm² without causing significant tissue damage, safety considerations for other peak wavelengths and corresponding tissue types may require lower irradiances, particularly in continuous wave applications. In certain embodiments, pulsed irradiances of light may be administered, thereby allowing safe application of significantly higher irradiances. Pulsed irradiances may be characterized as average irradiances that fall within safe ranges, thereby providing no or minimal damage to the applied tissue. In certain embodiments, irradiances in a range from 0.1 W/cm² to 10 W/cm² may be safely pulsed to target tissue.

Administered doses of light, or light doses, may be referred to as therapeutic doses of light in certain aspects. Doses of light may include various suitable combinations of the peak wavelength, the irradiance to the target tissue, and the exposure time period. Particular doses of light are disclosed that are tailored to provide safe and effective light for inducing one or more biological effects for various types of pathogens and corresponding tissue types. In certain aspects, the dose of light may be administered within a single time period in a continuous or a pulsed manner. In further aspects, a dose of light may be repeatably administered a number of times to provide a cumulative or total dose over a cumulative time period. By way of example, a single dose of light as disclosed herein may be provided over a single time period, such as in a range from 10 microseconds to no more than an hour, or in a range from 10 seconds to no more than an hour, while the single dose may be repeated at least twice to provide a cumulative dose over a cumulative time period, such as a 24-hour time period. In certain embodiments, doses of light are described that may be provided in a range from 0.5 joules per square centimeter (J/cm²) to 100 J/cm², or in a range from 0.5 J/cm² to 50 J/cm², or in a range from 2 J/cm² to 80 J/cm², or in a range from 5 J/cm² to 50 J/cm², while corresponding cumulative doses may be provided in a range from 1 J/cm² to 1000 J/cm², or in a range from 1 J/cm² to 500 J/cm², or in a range from 1 J/cm² to 200 J/cm², or in a range from 1 J/cm² to 100 J/cm², or in a range from 4 J/cm² to 160 J/cm², or in a range from 10 J/cm² to 100 J/cm², among other disclosed ranges. In a specific example, a single dose may be administered in a range from 10 J/cm² to 20 J/cm², and the single dose may be repeated twice a day for four consecutive days to provide a cumulative dose in a range from 80 J/cm² to 160 J/cm². In another specific example, a single dose may be administered at about 30 J/cm², and the single dose may be repeated twice a day for seven consecutive days to provide a cumulative dose of 420 J/cm².

In still further aspects, light for inducing one or more biological effects may include administering different doses of light to a target tissue to induce one or more biological effects for different target pathogens. Notably, light doses as disclosed herein may provide non-systemic and durable effects to targeted tissues. Light can be applied locally and without off-target tissue effects or overall systemic effects associated with conventional drug therapies which can spread throughout the body. In this regard, phototherapy may induce a biological effect and/or response in a target tissue without triggering the same or other biological responses in other parts of the body. Phototherapy as described herein may be administered with safe and effective doses that are durable. For example, a dose may be applied for minutes at a time, one to a few times a day, and the beneficial effect of the phototherapy may continue in between treatments.

Light sources may provide coherent light or incoherent light and may include one or more of LEDs, organic LEDs (OLEDs), lasers, and other lamps according to aspects of the present disclosure. Lasers may be used for irradiation in combination with optical fibers or other delivery mechanisms. LEDs are solid state electronic devices capable of emitting light when electrically activated. LEDs may be configured across many different targeted emission spectrum bands with high efficiency and relatively low costs. Accordingly, LEDs may be used as light sources in photonic devices for phototherapy applications. Light from an LED is administered using a device capable of delivering the requisite power to a targeted treatment area or tissue. High power LED-based devices can be employed to fulfill various spectral and power needs for a variety of different medical applications. LED-based photonic devices described herein may be configured with suitable power to provide irradiances as high as 100 mW/cm² or 200 mW/cm² in the desired wavelength range. An LED array in this device can be incorporated into an irradiation head, a hand piece, and/or as an external unit.

In addition to various sources of light, the principles of the present disclosure are also applicable to one or more other types of directed energy sources. As used herein, a directed energy source may include any of the various light sources previously described and/or an energy source capable of providing one or more of heat, IR heating, resistance heating, radio waves, microwaves, soundwaves, ultrasound waves, electromagnetic interference, electromagnetic radiation, and direct electrical stimulation that may be directed to a target body tissue. Combinations of visual and non-visual electromagnetic radiation may include peak wavelengths in a range from 180 nm to 4000 nm. Illumination devices as disclosed herein may include a light source and another directed energy source capable of providing directed energy beyond visible and UV light. In other embodiments, the other directed energy source capable of providing directed energy beyond visible and UV light may be provided separately from illumination devices of the present disclosure.

Illumination devices according to principles of the present disclosure include any devices configured to provide light therapy for promoting various health-related benefits. Exemplary illumination devices include those that are configured to prevent and/or treat infectious diseases (usable to stimulate an immune response to prevent infections, reduce the presence of pathogens, etc.), stimulate growth of hair, promote increased blood flow in the brain for the treatment of dementia, and/or modulate foreign body responses in tissue. In certain aspects, an illumination device may embody a device that is configured to direct light emissions within or through a body cavity, such as the oral cavity, to provide light treatments for upper respiratory infections. In other aspects, exemplary illumination devices may embody devices configured to provide light therapy to the scalp to promote hair growth or to provide light therapy to blood vessels associated with the brain. In still other aspects, exemplary illumination device may embody devices with probes and/or needles that remain in tissue for periods of time, such as continuous glucose monitors. Further devices may include lung light devices for delivering therapeutic doses to the lung for lower respiratory infections.

In certain aspects, illumination devices for providing phototherapy may embody connected devices that are part of larger systems that administer and/or monitor light treatment protocols across multiple illumination devices in one or more geographic locations. As used herein, treatment protocols may also be referred to as light treatment protocols or phototherapy protocols. Treatment protocols may include one or more wavelengths of light with corresponding dosing protocols that are intended to provide various biological effects. As used herein, such systems may include one or more servers that are able to communicate with individual illumination devices by way of one or more networks. In certain aspects, one or more local devices may serve as intermediate devices in communication with both the server and the individual illumination devices. In this manner, systems as described herein may allow monitoring of various phototherapy treatment protocols that are being administered in different geographic locations. The ability to compile geospatial information as illumination devices are being used may be beneficial in the early identification of infectious disease outbreaks. Notably, early detection may enable earlier implementation of safety measures in identified outbreak regions in order reduce outbreak severities. Additionally, when implemented phototherapy treatments in an outbreak region start to decline, the compiled geospatial information may enhance accuracy in determining when outbreaks are subsiding. In the context of other types of illumination devices, such as those that provide light therapy for hair growth or those that are used in tandem with other devices, such as continuous glucose monitoring, compiled geospatial information may provide other benefits, such as differentiating real-time health and/or wellness activities by geographic region.

Various elements associated with illumination devices and/or overall systems as described and/or illustrated herein may broadly represent any type or form of computing device or system capable of executing computer-readable instructions, such as those contained within the modules described herein. In their most basic configuration, these computing device(s) may each include at least one memory device and at least one physical processor.

In some examples, the term “memory device” generally refers to any type or form of volatile or non-volatile storage device or medium capable of storing data and/or computer-readable instructions. In one example, a memory device may store, load, and/or maintain one or more of the modules described herein. Examples of memory devices include, without limitation, random access memory (RAM), read only memory (ROM), flash memory, hard disk drives (HDDs), solid-state drives (SSDs), optical disk drives, caches, variations or combinations of one or more of the same, or any other suitable storage memory.

In some examples, the term “physical processor” generally refers to any type or form of hardware-implemented processing unit capable of interpreting and/or executing computer-readable instructions. In one example, a physical processor may access and/or modify one or more modules stored in the above-described memory device. Examples of physical processors include, without limitation, microprocessors, microcontrollers, central processing units (CPUs), field-programmable gate arrays (FPGAs) that implement softcore processors, application-specific integrated circuits (ASICs), portions of one or more of the same, variations or combinations of one or more of the same, or any other suitable physical processor.

Although various modules may be provided as separate elements, the modules described and/or illustrated herein may represent portions of a single module or application. In addition, in certain embodiments one or more of these modules may represent one or more software applications or programs that, when executed by a computing device, may cause the computing device to perform one or more tasks. For example, one or more of the modules described and/or illustrated herein may represent modules stored and configured to run on one or more of the computing devices or systems described and/or illustrated herein. One or more of these modules may also represent all or portions of one or more special-purpose computers configured to perform one or more tasks.

In addition, one or more of the modules described herein may transform data, physical devices, and/or representations of physical devices from one form to another. For example, one or more of the modules recited herein may receive sensor data to be transformed, transform the sensor data, output a result of the transformation to control impingement of light onto living tissue, use the result of the transformation to control impingement of light onto living tissue, and/or store the result of the transformation to control impingement of light onto living tissue. Additionally or alternatively, one or more of the modules recited herein may transform a processor, volatile memory, non-volatile memory, and/or any other portion of a physical computing device from one form to another by executing on the computing device, storing data on the computing device, and/or otherwise interacting with the computing device.

In certain embodiments, the term “computer-readable medium” generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., compact disks (CDs), digital video disks (DVDs), and Blu-ray disks), electronic-storage media (e.g., solid-state drives and flash media), and other distribution systems.

In certain aspects, servers are disclosed that are capable of sending and receiving information to and from multiple illumination devices that are in use. Servers may be capable of providing functionality and/or operating instructions for illumination devices in response to received information from illumination devices and/or receiving information from illumination devices once light treatments have been implemented. For example, servers may be capable of communicating treatment protocols to illumination devices based on received diagnostic information. Once light therapies have been implemented, illumination devices may be capable of communicating other information, such as location information, back to the server. In this regard, the server may be capable of compiling geospatial information related to timing and locations of implemented light treatments.

In the context of infectious diseases, such geospatial information may be beneficial in predicting outbreaks and/or for identifying regions where previously identified outbreaks are subsiding. The geospatial information may further be useful in identifying specific disease strains and/or variants that may be present in a certain area based on which treatment protocols are most prevalent. According to principles of the present disclosure, illumination devices and related systems for compiling geospatial information may beneficially provide precise light treatment information, or administered light treatment information, for use in evaluating various treatment protocols. For example, exact timings and dosing sequences of administered phototherapy may be captured by the system for comparison with patient outcomes. In this manner, instances where a patient does not actually receive phototherapy or instances where the patient does not complete all doses and/or days of a treatment protocol may be accounted for. In addition, a patient's physician may be informed of their patient's compliance with the treatment protocol. This information may improve patient outcomes by promoting compliance with prescribed treatments. In contrast, it may not always be clear if conventional medications are taken as prescribed, thereby making it difficult to accurately quantify efficacy in real world environments that are outside of clinical trials. For example, with a traditional pill, it is unknown if the patient actually ingested the pill or simply threw it away. In contrast, illumination devices according to the present disclosure may use sensors and/or cameras to confirm precise therapeutic dosing has been delivered. Providing verifiable dosing information that was delivered allows for more robust analysis of treatment protocols as described more fully below.

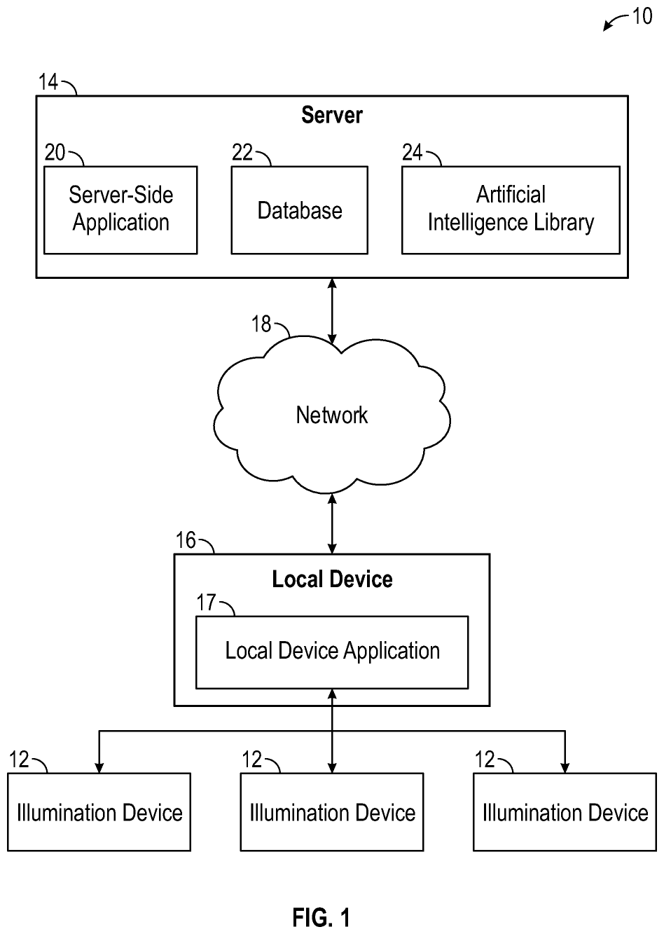

FIG. 1 is a schematic view of a system 10 for administering and monitoring phototherapy treatments of multiple illumination devices 12 at various geographic locations. In certain aspects, the illumination devices 12 may be separately controlled or managed by all or a portion the system 10. For example, the system 10 may include a server 14 in communication with one or more client-side or local devices 16 via a network 18. One or more local devices 16 may be associated with a single illumination device 12 or with multiple illumination devices 12 that reside in a common geographic location or region. Exemplary local devices 16 include, without limitation, laptops, tablets, desktops, local servers, cellular phones, personal digital assistants (PDAs), multimedia players, embedded systems, wearable devices (e.g., smart watches, smart glasses, etc.), routers, switches, gaming consoles, combinations of one or more of the same, or any other suitable computing device. In at least one example, the local device 16 may represent a user's computing device to which the user has paired with at least one illumination device 12. Additionally or alternatively, the local device 16 may include a local device application 17 for managing, controlling, and/or communicating with one or more of the illumination devices 12. The local device application 17 may embody an application on a computer or a mobile device, such as a phone, tablet, laptop, or wearable device, among others. In at least one embodiment, the local device application 17 may be configured to collect sensor data from one or more of the illumination devices 12 and/or user feedback that may be used by the server 14 and/or local device 16 to determine appropriate treatment protocols. In certain embodiments, the server 14 and the illumination devices 12 may be capable of communicating without intermediate local devices 16.

The server 14 may include a server-side application 20 for managing, controlling, and/or communicating with the local devices 16 and/or illumination devices 12. In at least one embodiment, the server-side application 20 may be configured to collect usage data associated with location information of the multiple illumination devices 12. The network 18 generally represents any medium or architecture capable of facilitating communication or data transfer. Examples of the network 18 include, without limitation, an intranet, a wide area network (WAN), a local area network (LAN), a personal area network (PAN), the Internet, power line communications (PLC), a cellular network (e.g., a global system for mobile communications (GSM) network), or the like. The network 18 may facilitate communication or data transfer using wireless or wired connections between the server 14 and the one or more local device 16 and the illumination devices 12.

In certain embodiments, the server 14 may include a database 22 and/or an artificial intelligence library 24 that are populated with suitable data, including but not limited to clinical trial data and data (e.g., images and other sensor data) captured by other illumination devices in practice, that allows the server-side application 20 to receive data specific to a particular user, compare the data with the artificial intelligence library 24, and formulate a tailored phototherapy treatment protocol. The artificial intelligence library 24 may be continually updated and refined based on populated data to continuously improve the ability of the server-side application 20 to provide malady detection and corresponding tailored phototherapy treatments with increased efficacy. As used herein, the artificial intelligence library 24 may refer to a collection of data (e.g., images and/or sensor data) that correspond to previously identified characteristics of body tissues, including but not limited to the presence of pathogens, diseases, cancerous or pre-cancerous lesions, tumors or polyps, accumulation of fluid, and inflammation, among other tissue characteristics and conditions. In this manner, the artificial intelligence library 24 may be utilized by the server-side application 20 and/or the local device application 17 to recognize diagnostic information received from the illumination devices 12, compare the received diagnostic information to data received from other illumination devices, and provide appropriate treatment protocols that may be administered by the illumination devices 12 for inducing any number of biological effects.

According to principles of the present disclosure, the system 10 may provide a method that includes accessing data related a particular user, generating at least one treatment protocol based on the data, communicating the treatment protocol to the illumination device(s) 12 associated with the user, and providing geospatial location information related to the implemented treatment protocol. Compiled geospatial location information from multiple users and multiple illumination devices may accordingly be used to provide overall geographical information.

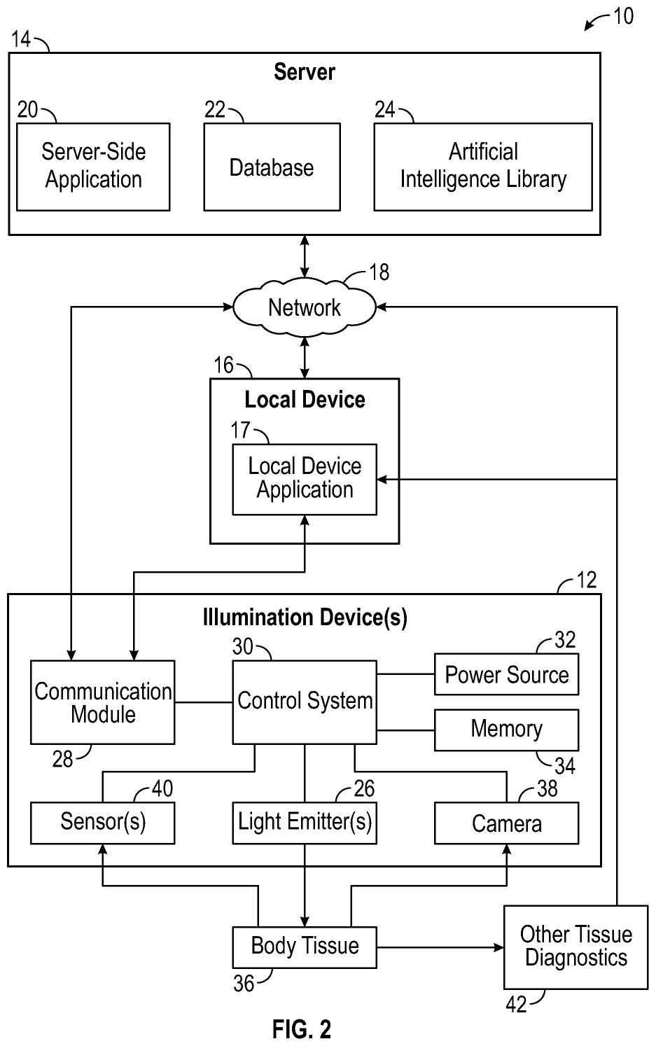

FIG. 2 is a schematic view of the system 10 of FIG. 1 with further details provided for one illumination device 12. While a single illumination device 12 is represented in FIG. 2, it is understood that principles described are applicable to any of the illumination devices 12 that may be associated with the system 10. The illumination device 12 may include one or more light emitters 26, a communication module 28, and a control system 30 associated with the light emitters 26 and the communication module 28. The communication module 28 may facilitate communication with the local device 16 and the local device application 17. The communication module 28 may also be configured to communicate directly with the server 14 by way of the network 18 and without the intermediate local device 16. The communication module 28 may provide communication via any number of manners, including Bluetooth, wired and/or wireless internet connections, a cellular network, analog communication such as one or more pre-programmed buttons of the illumination device 12, or any other form of analog or digital communication. The control system 30 may include emitter driving circuitry, among other control circuitry, that is configured to drive the light emitters 26 according to a treatment protocol. The control system 30 may further be configured to determine location information associated with the implemented treatment protocol and send the location information to the server 14 by way of the communication module 28.

The illumination device 12 may include a power source 32 that includes any type of internal power source and/or connections to an external power source. For example, the power source 32 may embody a portable power source and/or an energy storage device that is provided within the illumination device 12, such as a replaceable battery and/or a rechargeable battery. For rechargeable embodiments, the illumination device 12 may include a port, (e.g., a universal serial bus port, a power plug, or the like) for providing a connection to an external power source or even another device, such as the local device 16, for recharging. In certain embodiments, the port may also facilitate data transfer and communication via the communication module 28. The power source 32 may be configured for direct connections to an external power source with or without recharging capabilities, including a wired and/or a plug-direct configuration to the external power source. As used herein, the external power source may include a hardwired electrical connection such as a wall plug or any type of wired or portable external energy storage device. In still further embodiments, the external power source coupled to the power source 32 of the illumination device 12 may embody a human factor power source at the client-side that provides power responsive to human movements, such as walking and/or chewing by a user. The external power source may further embody renewable energy sources, including solar and/or wind sources, that provide power to and or recharging of the power source 32. In certain applications, the system 10 may include a solar element or panel that may be worn by a user of the illumination device 12, such as solar hat, a solar sleeve, or any other form of solar clothing.

In certain aspects, the illumination device 12 may include a memory device 34 that stores various drive algorithms and/or control schemes for the control system 30 based on information, including information received from the server 14. The memory device 34 may further be configured to store data and diagnostic information collected at a body tissue 36 of a user for communication with the server 14. As described above, the memory device 34 may include any type or form of a volatile and/or a non-volatile storage device or any medium capable of storing data and/or computer-readable instructions. For example, the memory device 34 may include, without limitation, RAM, ROM, flash memory, HDDs, SSDs, optical disk drives, caches, and variations or combinations of one or more of the same, or any other suitable storage memory. While the control system 30, the communication module 28, and the memory device 34 are illustrated as separate blocks or elements, each of the control system 30, the communication module 28, and the memory device 34 may also embody elements within a combined overall control circuitry module for the illumination device 12.

In certain applications, the illumination device 12 may include one or more of a camera 38 and one or more sensors 40 configured for capturing images or other diagnostic information of the body tissue 36 that may be relayed back to the server 14 for analysis. Captured images may include one or more visible-light images, one or more IR images, one or more UV images, one or more images measuring light within a predetermined range of wavelengths, one or more images measuring light within two or more different predetermined ranges of wavelengths, reflected resonance images, reflected wave images, and ultrasound images. The sensors 40 may include one or more of temperature sensors, photo sensors, image sensors, proximity sensors, blood pressure or other pressure sensors, chemical sensors, biosensors (e.g., heart rate sensors, body temperature sensors, sensors that detect presence or concentration of chemical or biological species, or other conditions), accelerometers, moisture sensors, oximeters, such as pulse oximeters, current sensors, voltage sensors, and the like. The camera 38 and sensors 40 may work together as needed to perform various functions, including identifying a location of a launch lens or plane relative to a disease location of the body tissue 36, including but not limited to various tissues, suspended mucous, hardened puss pockets, organs, and bones. The camera 38 may further provide precise location information for the body tissue 36 based on camera pixelated measurements and global positioning system (GPS) data, among others.

In still further embodiments, the camera 38 and/or sensors 40 may capture three-dimensional imaging, such as light detection and ranging (LIDAR) of one or more portions of the user, including the body tissue 36 or other body portions associated with positioning the illumination device 12 relative to the body tissue 36. Such three-dimensional images may be relayed back to the server 14 for determining specific and/or custom arrangements of the illumination device 12. In certain aspects, the three-dimensional images may be used to choose between various pre-configurations of the illumination device 12 as part of a treatment protocol. In other embodiments, the three-dimensional images may be used to create custom shapes for certain elements of the illumination device 12. For example, with precise three-dimensional imaging of a user's oral cavity, a custom mouthpiece may be generated and sent to the user for use during phototherapy in or through the oral cavity. Additionally, the local device 16 may be in communication with a three-dimensional printer that may create the custom mouthpiece for more immediate use.

In combination with or in place of images and/or other diagnostic information that may be collected by the illumination device 12, the system 10 may also be configured to receive other tissue diagnostics 42 that are collected separately from the illumination device 12. The other tissue diagnostics 42 may include external cameras and sensors that are similar to the any of the above-described embodiments of the sensor 40 and the camera 38. Additionally, the other tissue diagnostics 42 may be collected by any number of other medical devices, including ultrasounds, x-ray, magnetic resonance imaging, and the like. In further embodiments, the other tissue diagnostics 42 may include information provided by a user and/or a medical professional based on a physical examination and/or diagnostic tests administered to the body tissue 36 and the corresponding user.

The captured images and/or sensor data from the illumination device 12 and/or provided by the other tissue diagnostics 42 may be relayed to one or more of the local device 16 and/or the server 14 for analysis. Accordingly, the captured images and/or data may be compared with large volumes of photos of known diseased tissue and corresponding data that are stored in the artificial intelligence library 24. In this regard, the system 10 may determine characteristics of the body tissue 36 including but not limited to one or more of a name and strain of one or more pathogens that are present, a size of an impacted area of the body tissue 36, any cancerous or pre-cancerous lesions, tumors or polyps, accumulation of fluid, and inflammation. In certain embodiments, the illumination device 12 may be configured to administer multiple wavelengths of light for inducing multiple biological effects, either concurrently or sequentially based on the treatment protocol. For example, the illumination device 12 may detect that an initial dose from the treatment protocol resulted in inflammation or tissue damage. Based on this information, the illumination device 12 may then provide a second dose of light at a different wavelength from the initial dose that treats tissue damage and/or reduces inflammation. In a specific example, a dose of red light could be provided to repair tissue after a dose of UV light has been provided to eliminate pathogens but may have damaged the tissue. The artificial intelligence library 24 may initially be populated with as many images as possible that are then added to with each subsequent new patient data. This provides the system 10 with the ability to expand and evolve for improved malady identification so that appropriate and up-to-date treatment protocols may be delivered to the body tissue 36. The system 10 may further provide functionality that includes determining corresponding treatment costs to provide real-time billing, appropriate insurance claims, and exchange of payments. In certain embodiments, the system 10 may further be used to monitor the body tissue 36 and recommend a subsequent anti-inflammatory treatment depending on the resolution of the disease.

In this manner, patient outcomes may continually be optimized by the server 14 based on collective information received by multiple ones of the illumination devices 12 across a large volume of different body tissues. Optimization may refer to a best-available or a continually-improved medical outcome such as one or more of prevention, treatment, cure, and follow-up treatments for one or more conditions that may be present. The server 14 may further identify other recommended treatments for the body tissue 36 that may be implemented in combination with the illumination device 12, such as one or more medications that may be administered to further improve or optimize medical outcomes.

The treatment protocol provided by the server 14 may include any number of changeable attributes for the illumination device 12, such as one or more peak wavelengths, radiant fluxes, irradiances, exposure times, and corresponding doses that may be provided by the light emitters 26 to the body tissue 36. Treatments may be administered over any time range as previously described, including by way of example, a range of 0.05 to 360 seconds of total illumination device 12 operation per treatment or dose. Doses may be provided by a series of energy sources or alternatives of the same energy source (e.g., different peak wavelengths of light) that may be deployed in a singular or multiple fashion according to any of the previously described embodiments. As described herein, treatments and/or doses may be provided with appropriate safety, efficacy, and time per treatment for achieving the best possible outcomes in fighting one or more targeted pathogens, diseases, or other conditions.

In certain embodiments, one or more of the light emitters 26 may provide changeable attributes from visible light sources such as one or more of LEDs, OLEDs, incandescent light sources, fluorescent light sources, liquid crystal displays, lasers, halogen sources, tungsten-halogen sources, sodium vapor sources, gas laser sources, microwave photons, biological sources such as dinoflagellates, and light that is harnessed from sunlight, including filtered and unfiltered sunlight. In certain embodiments, the one or more light emitters 26 may include light sources beyond just visible light, including but not limited to UV light sources, quick-flash UV-C light or other rapid UV emissions from any suitable UV light sources, and IR sources. While previously described embodiments have been provided in the context of various sources of light, the principles of the present disclosure are also applicable to one or more other types of directed energy sources. As used herein, a directed energy source may include any of the various light sources previously described, and/or an energy source capable of providing one or more of heat, IR heating, resistance heating, radio waves, microwaves, soundwaves, ultrasound waves, electromagnetic interference, and electromagnetic radiation that may be directed to the body tissue 36. In certain embodiments, changeable attributes provided by the server 14 may include protocols for administering any of the directed energy sources listed above to the body tissue 36. For example, the illumination device 12 may include one or more directed energy sources capable of providing directed energy beyond visible and UV light to the body tissue 36, alone or in combination with the light emitters 26. In other embodiments, the directed energy source capable of providing directed energy beyond visible and UV light may be provided separately from the illumination device 12 while still being in communication with the server 14 in a similar manner as described for the illumination device 12. The changeable attributes may also include identification of one or more combinations of optics, locators, light source positioners, and light guide positioners that may be attached or otherwise utilized by the illumination device 12 to deliver identified doses of light to different types of the body tissue 36, such as one or more tissues of the upper respiratory tract, the auditory canal, the nasal cavity, the oral cavity, the oropharyngeal area, the throat, the larynx, the pharynx, the oropharynx, the trachea, the esophagus and the like, to stimulate mucosal epithelial cells. In further embodiments, the body tissue 36 may also include tissues of one or more of the lungs and endothelial tissues. In still further embodiments, the body tissue 36 may also include any subordinate areas related to respiratory diseases, including gastrointestinal tissue that processes mucous. In still further embodiments, the body tissue 36 may include the skin and/or scalp of a user.

According to further implementations, any of the above-described elements and functions of the system 10 may be provided with less automated configurations. For example, a more simplified version of the system 10 may include a configuration where a user may click-through a menu or simply press pre-configured buttons on the illumination device 12 and/or the local device 16 to select a particular treatment program. In another example, a user may progress through one or more steps on the illumination device 12 and/or the local device 16 to provide images or other diagnostic information via the illumination device 12 or via off-the-shelf test kits or other in-office procedures. In certain embodiments, one or more of the local device 16 and the illumination device 12 may also include a local artificial intelligence library so that treatment protocols may be provided without having to first communicate with the server 14. In such embodiments, the local artificial intelligence library may be periodically synchronized with the artificial intelligence library 24 of the server 14 according to routine intervals.

In any of the above-described embodiments, the system 10 may be well-suited for communicating implemented treatment protocols associated with geographic locations to the server 14. Accordingly, the server 14 may be capable of compiling geospatial information related to timing and locations of implemented light treatments. In the context of infectious diseases, such geospatial information may be beneficial in predicting outbreaks, identifying locations of disease variants, and/or for identifying regions where previously identified outbreaks are subsiding.

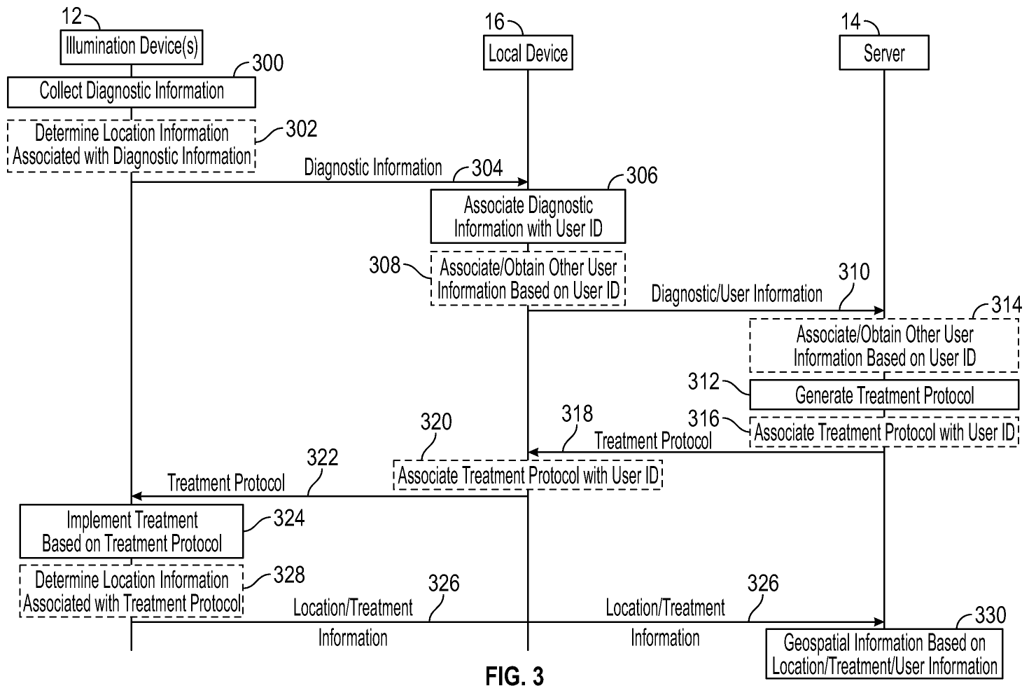

FIGS. 3 to 6 are call-flow diagrams illustrating various implementations of the system 10 of FIGS. 1 and 2. Each of the call-flow diagrams generally lists the illumination device(s) 12, the local device 16, and the server 14 as described above for FIGS. 1 and 2. In this regard, the local device 16 and the server 14 as illustrated in FIGS. 3 to 6 may form part of the overall system 10 along with any of the other above-described elements of the system 10 described above for FIGS. 1 and 2. In each of FIGS. 3 to 6, dashed line boxes may indicate optional portions of the call-flow diagram and/or different stages of the call-flow diagrams where certain steps may be performed. While functionality may be illustrated that is associated with one or more combinations of the illumination device(s) 12, the local device 16, and the server 14, it is understood that FIGS. 3 to 6 represent exemplary embodiments. In this manner, functionality that is shown in an associated manner with one of the illumination device(s) 12, the local device 16, or the server 14 may alternatively be implemented by a different one of the illumination device(s) 12, the local device 16, and the server 14, or combinations thereof.

FIG. 3 is a call-flow diagram illustrating an implementation of the system 10 of FIGS. 1 and 2 where the server 14 determines a treatment protocol for the illumination device 12 based on received diagnostic and/or user information from the illumination device 12 and/or the local device 16. For example, the illumination device 12 may collect diagnostic information (step 300) associated with a user based on imaging and/or sensor data collected by the illumination device 12 and/or other diagnostic information collected external to the illumination device 12. In certain embodiments, the local device 16 may then determine location information (step 302) associated with the diagnostic information, while in other embodiments, the location information may be associated at a later step as described below. The illumination device 12 may then send the diagnostic information (step 304) to the local device 16. In certain embodiments, the local device 16 may associate additional information (step 306), such as a user identification (ID) with the diagnostic information. With the user ID, the local device 16 may accordingly obtain and associate other user information, such as the user's medical history and/or the user's demographics with the diagnostic information (step 308). The local device 16 may then send the diagnostic information and the user information to the server 14 for analysis (step 310). In other embodiments, the local device 16 may serve as a pass-through for the diagnostic information without associating the user ID.

The server 14 may be configured to receive the diagnostic information and user information and then generate a specific treatment protocol for the user (step 312). In certain embodiments, the server 14 may associate the user ID and/or other user information with the received diagnostic information (step 314), particularly when this step is not performed by the local device 16. In still further embodiments, the association of the user ID and/or other user information may be omitted. When the user ID is present, the server 14 may optionally associate the generated treatment protocol with the user ID (step 316) before sending the treatment protocol (step 318) to the local device 16. In other embodiments, the optional step of associating the generated treatment protocol with the user ID may be performed at the local device 16 after the local device 16 receives the treatment protocol (step 320). The local device 16 may then send the treatment protocol to the illumination device 12 (step 322). The illumination device 12 may then implement the treatment protocol (step 324) and send the completed treatment information along with the location information back to the server 14 (step 326). As used herein, completed treatment information, which may also be referred to as administered light treatment information, refers to information related to actual light treatments received by a user. Such information may include dosing information, including wavelengths and/or timing of implemented light treatments. In this regard, administered light treatment information may be helpful for evaluating efficacy of light treatments by differentiating intended treatment protocols from light treatments that were actually administered to a user. In certain embodiments, the treatment protocol received from the server 14 may be modified from a pre-configured protocol that is loaded on the illumination device 12. For embodiments where the location information has not already been associated with the treatment protocol, the local device 16 may associate the location information after implementing treatment (step 328). Finally, the server 14 may compile geospatial information based on received treatment, location, and user information to provide geographical information related to usages of light therapy as described above (step 330).

In certain embodiments, the illumination device 12, the local device 16, and the server 14 may perform the above steps in a continuous or real-time basis. For example, the illumination device 12 and/or the local device 16 may provide the diagnostic information and/or the user information to the server 14 while light treatments are being administered. The server 14 may accordingly adjust the treatment protocol during the light treatment in response to collected diagnostic information as tissue receives the light treatment. In certain aspects, such real-time exchange of information may allow the treatment protocol to be adjusted based on a detected size of the treatment area and/or a detected topography of the treatment area to provide tailored light treatments in a safe and effective manner. In still further embodiments, such real-time exchange of information may allow monitoring of various operating functions of the illumination device 12, such as battery or charging errors, or light output, that may trigger a software patch that may be downloaded to the illumination device 12 or even a replacement or recall of the illumination device 12.

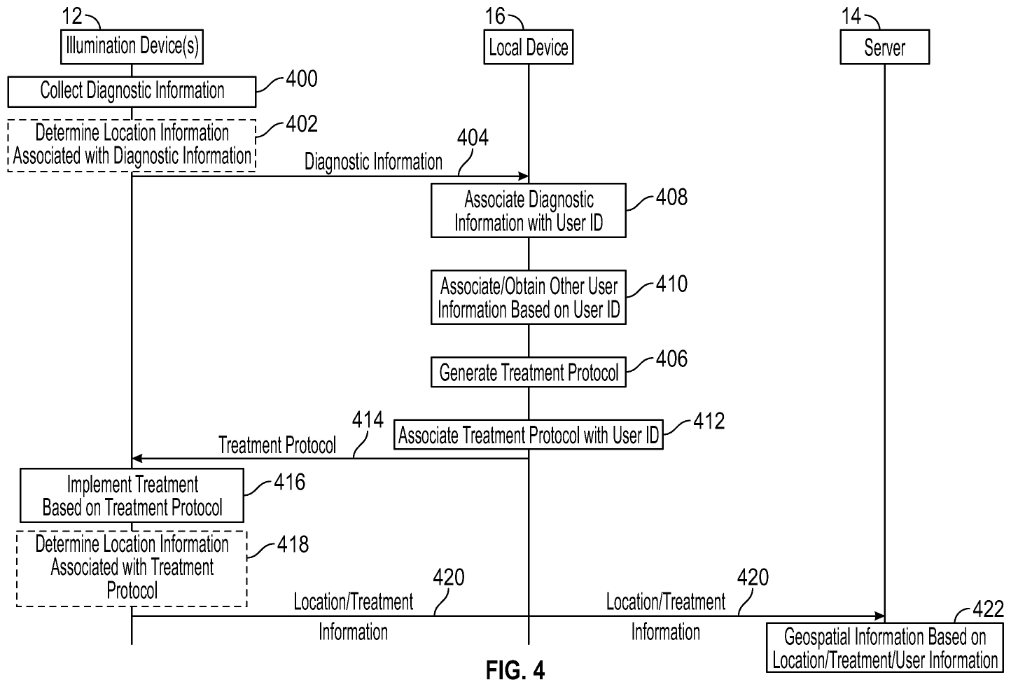

FIG. 4 is a call-flow diagram illustrating another implementation of the system 10 of FIGS. 1 and 2 where the local device 16 determines a treatment protocol for the illumination device 12 and the implemented treatment and location information is sent to the server 14. In this manner, the local device 16 may be configured to provide much of the functionality described for the server 14 in FIG. 3. As illustrated, the illumination device 12 may collect diagnostic information (step 400), and optionally determine location information associated with the diagnostic information (step 402) before sending the diagnostic information (step 404) to the local device 16. The local device 16 may receive diagnostic information and optionally the location information from the illumination device 12 and then generate the treatment protocol (step 406). As with FIG. 3, the local device 16 may also associate the diagnostic information with the user ID (step 408), obtain any other user information that may be associated with the user ID (step 410), and associate the user ID with the generated treatment protocol (step 412). The local device 16 may then send the treatment protocol (step 414) to the illumination device 12 for implementation (step 416). If the location information has not already been associated, the illumination device 12 may do after or concurrently with implementation (step 418). The illumination device 12 may then send the completed or administered treatment information along with the location information back to the server 14 (step 420) for geospatial compiling (step 422). The administered treatment information may also include one or more of the diagnostic information for a user and additional user information such as a user's medical history and/or a user's demographics. For embodiments where the location information has not already been associated with the treatment protocol, the local device 16 may associate the location information after implementing treatment. Upon receiving the location and administered treatment information from multiple ones of the illumination devices 12, the server 14 and/or the server-side application 20 may compile geospatial information or provide geospatial information for compiling by another device that can access the geospatial information.

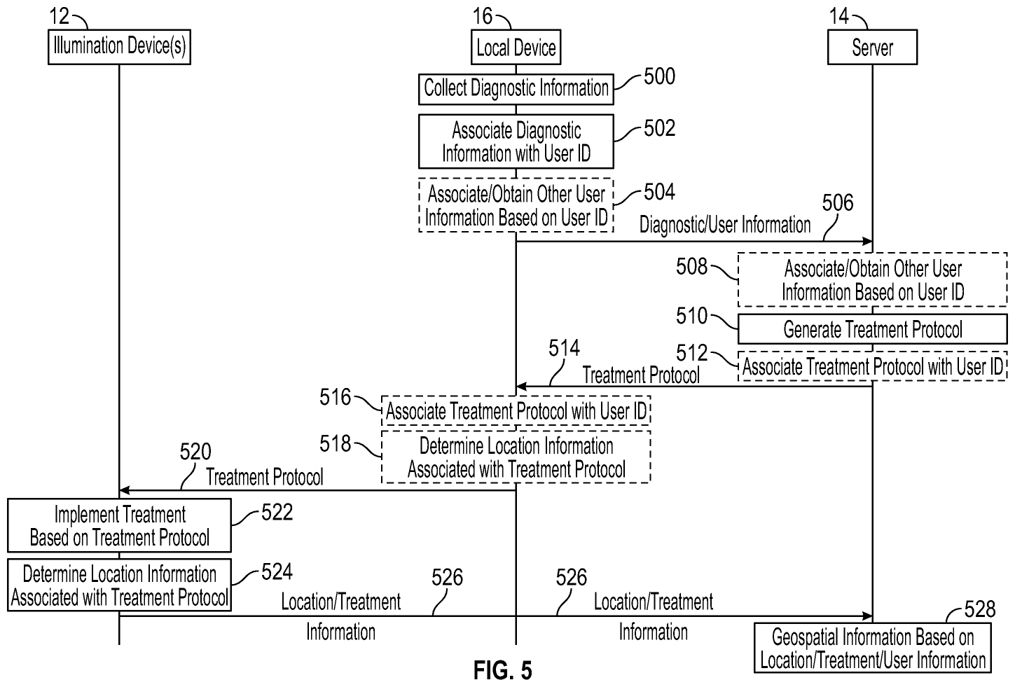

FIG. 5 is a call-flow diagram illustrating another implementation of the system 10 of FIGS. 1 and 2 where the local device 16 collects diagnostic information (step 500) and associates the diagnostic information with a user ID (step 502) independently from the illumination device 12. The local device 16 may further obtain other user information based on the user ID (step 504), or this step may take place at the server 14. The local device 16 communicates the diagnostic and user information to the server 14 (step 506) and receives the treatment protocol from the server 14 as described above. The server 14 may associate the other user information based on the user ID (step 508), generate the treatment protocol (step 510) and associate the generated treatment protocol with the user ID (step 512) before sending the treatment protocol to the local device 16 (step 514). In such a configuration, diagnostic information may be provided to the local device 16 from the user or from a health professional who has evaluated the user and collected various imaging and/or other testing information. As with other embodiments, the local device 16 may associate the treatment protocol with the user ID (step 516) and determine location information associated with the treatment protocol (step 518) before sending the treatment protocol to the illumination device 12 (step 520). In this manner, the illumination device 12 may be configured to receive and implement treatment protocols (step 522) and optionally determine location information associated with the treatment protocol (step 524) before sending the completed treatment information and location information to the server 14 (step 526) for geospatial compiling (step 528).

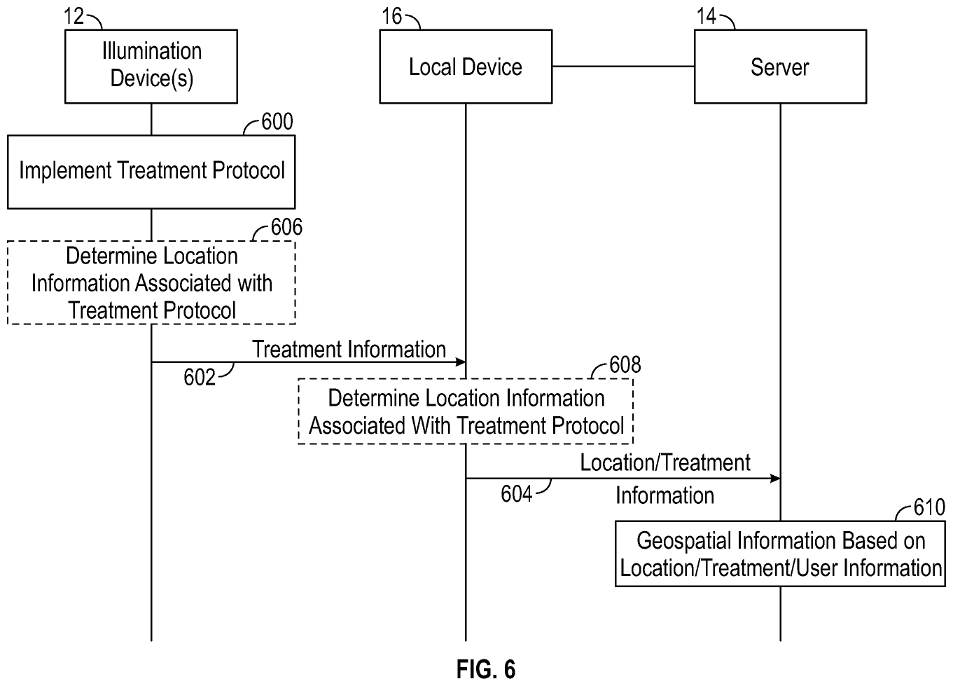

FIG. 6 is a call-flow diagram illustrating another implementation of the system 10 of FIGS. 1 and 2 where the illumination device 12 is pre-configured with one or more treatment protocols that may be implemented. The one or more pre-configured treatment protocols may be loaded or programmed with the control system 30 of the illumination device 12. In this manner, a user may self-administer the pre-configured treatment protocol based on symptoms or at the recommendation of a health professional (step 600). In certain aspects, the illumination device 12 may be configured to modify the pre-configured treatment protocol based on diagnostic information collected by way of a camera and/or a sensor that is associated with the illumination device 12. After light treatments are administered, the illumination device 12 may send the administered treatment information to the local device 16 (step 602) and the server 14 (step 604). Either the illumination device 12 or the local device 16 may determine the location information associated with the implemented treatment protocol (step 606 or step 608). Upon receiving the location and administered treatment information from multiple illumination devices 12, the server 14 and/or the server-side application 20 may compile geospatial information or provide geospatial information for compiling as described above (step 610).

As described above in the context of the system 10 of FIGS. 1 and 2 and any of the call-flow diagrams illustrated in FIGS. 3 to 6, various types of information are exchanged between one or more of the illumination devices 12, local devices 16, networks 18, and servers 14. Such information may include diagnostic information associated with a user, location information associated with the user, additional user information including a user ID and a medical history of the user, treatment protocols, and/or location information associated with implemented treatments. In view of the sensitive nature of such information, certain embodiments involve one or more of the illumination devices 12, local devices 16, networks 18, and servers 14 being configured to send and/or receive information that is protected by way of encryption or other protection techniques. For example, one or more of the illumination devices 12, local devices 16, networks 18, and servers 14 may be configured to send and/or receive encrypted data. In another example, one or more of the illumination devices 12, local devices 16, networks 18, and servers 14 may be configured to send and/or receive information based on blockchain technology. For blockchain technology, the server 14 and database 22 as illustrated in FIG. 2 may be decentralized across multiple devices according to distributed ledger technology. In this regard, sensitive information related to the user, the user's location, and the implemented treatment protocol may be readily exchanged with enhanced digital security.

Illumination devices 12 for the system 10 of FIGS. 1 and 2 and for any of the call-flow diagrams illustrated in FIGS. 3 to 6 may embody various device types configured to induce various biological effects and/or promote various health-related benefits. Exemplary illumination devices include those that are configured to treat infectious diseases, to stimulate growth of hair, to stimulate increased blood flow in the brain for the treatment of dementia, and/or to modulate foreign body responses in tissue.

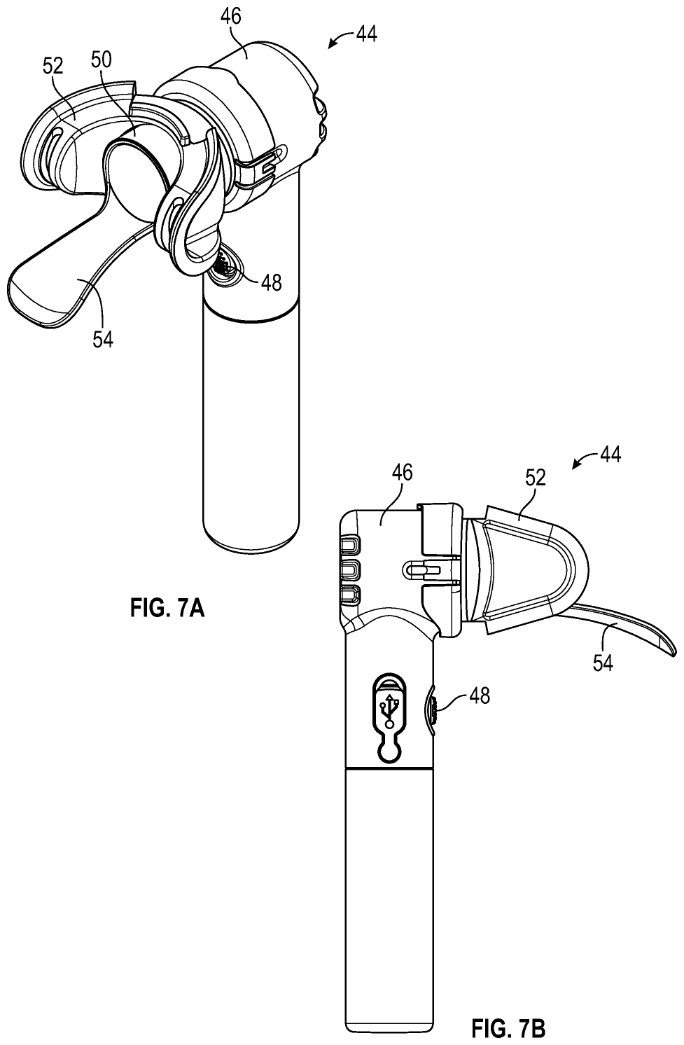

FIG. 7A is a perspective view of an exemplary illumination device 44 that is configured to direct light emissions within or through a body cavity, such as the oral cavity. FIG. 7B is a side view of the illumination device 44 of FIG. 7A. The illumination device 44 may embody a handheld device for delivering light (e.g., nitric-oxide modulating light and/or light to induce any of the previously described biological effects) to living tissue within or near a user's oral cavity, including the oropharynx. For infectious diseases of the upper respiratory system, initial infection sites may be associated with tissue of the oropharynx. In this manner, the ability of the illumination device 44 to provide directed emissions to the oropharynx may provide inactivation and/or reduced replication of pathogens at early stages of infection. The illumination device 44 includes a housing 46 for containing and protecting one or more light emitters as previously described. The housing 46 may also include the communication module 28 and the control system 30, among other elements as illustrated in FIG. 2. A button 48 may be provided along the housing 46 for energizing the illumination device 44 and/or the internal light emitter(s). The illumination device 44 may include a light guide 50 and a light-guide positioner 52 suitably sized and shaped for insertion into a user's oral cavity. The light guide 50 may embody a hollow light guide through with light travels, while in other embodiments, the light guide 50 may embody a material through which the light propagates. In certain aspects, the light-guide positioner 52 may be referred to as a mouthpiece for the illumination device 44. A portion of the light guide 50 may form a tongue depressor 54 that is configured to depress a user's tongue when inserted into the user's mouth to provide a direct light path from the light guide 50 to the oropharynx.

In the context of the call-flow diagrams of FIGS. 3 to 6, multiple illumination devices 44 may be implemented as the illumination devices 12 in FIGS. 3 to 6. In the treatment of infectious diseases of the upper respiratory system, such as Orthomyxoviridae (e.g., influenza) and Coronaviridae (e.g., SARS-CoV-2), among others, different treatment protocols may be developed in the treatment of various diseases and/or strains or variants of a particular disease. In certain aspects, targeted treatment protocols may be varied for individual illumination devices 44 based on collected diagnostic information, such as sensor and/or camera data. In the context of targeting the oropharynx through the oral cavity, collected diagnostic data may identify a larger distance from the front of the mouth to the oropharynx based on a particular user's anatomy. With this information, a treatment protocol may be developed that increases a time and/or intensity of light emissions so the user may receive the targeted dose. In further aspects, the collected diagnostic data may be used to determine actual dosing received by the user and reported to the server 14. For example, a treatment protocol may be developed based on a user's symptoms and implemented by the illumination device 12. The illumination device 12 may associate collected diagnostic data at the time of treatment that includes the actual distance to the oropharynx. In this regard, the administered light treatment information reported back to the server 14 may include an actual received dose that accounts for variations based on the user's anatomy. With this information, the server 14 would have actual doses administered and in combination with outcomes of the user, the server 14 may be able to adjust treatment protocols for other users with increased precision. In this regard, the compiling of geospatial data as described above may be useful in providing early detection of regional outbreaks, based on which treatment protocols are most prevalent.

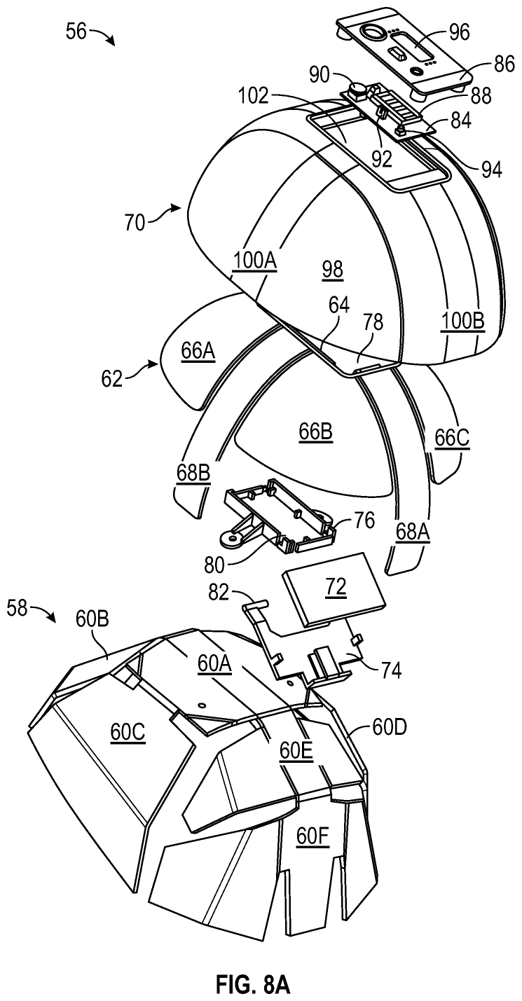

FIGS. 8A-8B illustrate an exemplary illumination device 56 that is configured to provide light therapy to the scalp and/or brain of a patient to promote hair growth or to promote increased blood flow in the brain for the treatment of dementia. In the context of the call-flow diagrams of FIGS. 3 to 6, multiple illumination devices 56 of FIG. 8A may be implemented as the illumination devices 12 in FIGS. 3 to 6. In the treatment of hair regrowth of the scalp or in the treatment of blood vessels associated with the brain, the compiling of geospatial data as described above may be useful in providing regional demographics based on what treatment protocols are most used and/or of greatest interest.

FIG. 8A is an exploded view of the illumination device 56 embodied as a wearable cap for delivering phototherapy to a scalp and/or brain of a user. The illumination device 56 may include multiple light emitters and standoffs supported by a flexible printed circuit board (FPCB) 58 including multiple interconnected panels 60A-60F arranged in a concave configuration. A concave shaping member 62 (including a frame 64, ribs 66A-66C, and curved panels 68A-68B) is configured to receive the FPCB 58. The ribs 66A-66C and curved panels 68A-68B project generally outwardly and downwardly from the frame 64. Gaps are provided between portions of adjacent ribs 66A-66C and curved panels 68A-68B to accommodate outward expansion and inward contraction, and to enable transfer of heat and/or fluid (e.g., evaporation of sweat). A fabric covering element 70 is configured to cover the concave shaping member 62 and the FPCB 58 contained therein. A battery 72 and a battery holder 74 are arranged between the FPCB 58 and the concave shaping member 62. An electronics housing 76 is arranged to be received within an opening 78 defined in the frame 64 of the concave shaping member 62. Pivotal coupling elements 80, 82 are arranged to pivotally couple the battery holder 74 to the electronics housing 76. An electronics board 84 is insertable into the electronics housing 76, which is enclosed with a cover 86. Various elements may be arranged on the electronics board 84, such as a cycle counter 88, a control button 90, a charging/data port 92, and a status lamp 94. The various elements associated with the electronics housing 76 and the electronics board 84 may be referred to generally as a “control module.” Windows 96 may be defined in the cover 86 to provide access to the cycle counter 88, the control button 90, the charging/data port 92, and the status lamp 94. The fabric covering element 70 includes a fabric body 98 and multiple internal pockets 100A-100B arranged to receive portions of the ribs 66A-66C. An opening 102 at the top of the fabric covering element 70 is arranged to receive the cover 86.

FIG. 8B is a bottom plan view of the FPCB 58 of FIG. 8A illustrating light emitters 104 and standoffs 106 arranged thereon. The FPCB 58 may include a polyimide substrate 107 with an inner surface 107A configured to conform in a concave shape. In one embodiment, the light emitters 104 may include a total of 280 LEDs arranged as 56 strings of 5 LEDs, with a string voltage of 11 volts (V), a current limit of 5 milliampere (mA), and a power consumption of 3.08 watts (W). FIG. 8B illustrates 36 standoffs 106 extending from the inner surface 107A of the FPCB 58. The FPCB 58 may include six interconnected panels 60A-60F, with the panels 60A-60F being connected to one another via narrowed tab regions. Gaps are formed between various panels 60A-60F to allow the FPCB 58 to conform in the shape of a user's head and to permit transport of heat and/or fluid (e.g., evaporation of sweat) between the panels 60A-60F.

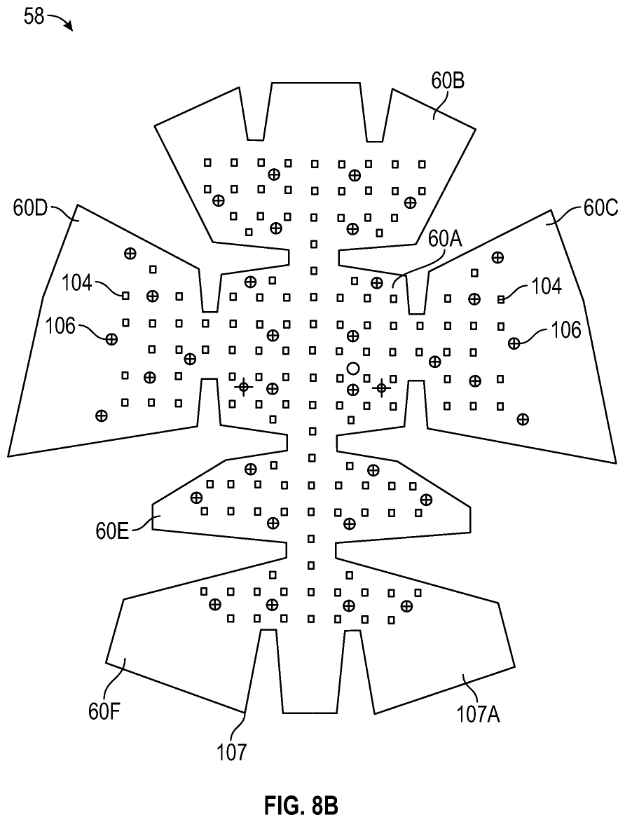

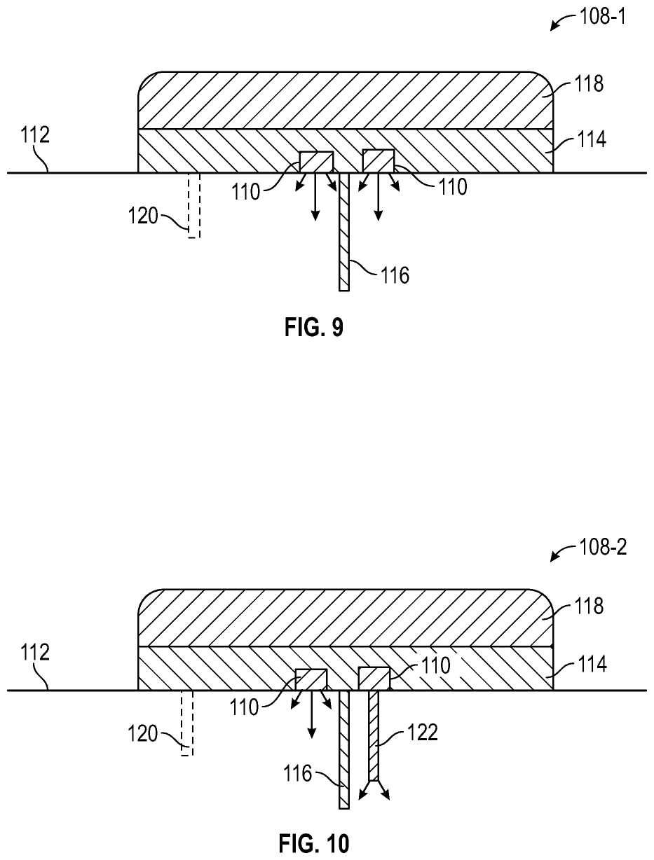

FIGS. 9 and 10 illustrate exemplary illumination devices 108-1 and 108-2 that may embody devices with probes and/or needles that remain in tissue for periods of time, such as continuous glucose monitors (CGMs). In the context of the call-flow diagrams of FIGS. 3 to 6, multiple illumination devices 108-1, 108-2 may be implemented as the illumination devices 12 in FIGS. 3 to 6 to provide geospatial information as described above.

FIG. 9 is an illustration representing a CGM 108-1 with an incorporated light source 110 capable of delivering FBR-modulating light to a host's skin 112 during monitoring. The light source 110 may embody any of the light emitters 26 as previously described for FIG. 2. The CGM 108-1 may generally include a sensor holder 114 that includes a sensor probe 116. The sensor holder 114 may mechanically support the sensor probe 116 during percutaneous insertion. In certain configurations, the sensor probe 116 may be provided in a perpendicular manner relative to the CGM 108-1. However, in other configurations, the sensor probe 116 may be provided at an angle relative to the CGM 108-1. The sensor holder 114 is typically secured to the skin 112 by way of an adhesive. The CGM 108-1 may further include a transmitter 118 capable of relaying glucose sensing information to an external device, such as one or more of a portable monitor, a cell phone, a wearable device (e.g., a watch or other graphical display device), a computer, and a network. In this manner, the external device may embody one or more of the local device 16 and the server 14 of FIGS. 1 and 2. The transmitter 118 may include one or more of a power source (e.g., a battery or rechargeable battery), a microprocessor and/or microcontroller, a communications module (e.g., 28 of FIG. 2) for facilitating wireless and/or wired communications, and other associated electronics. In still further embodiments, the CGM 108-1 may further include an optional insulin infusion catheter 120. In other embodiments, an associated insulin infusion catheter may be provided separately from the CGM 108-1. As illustrated in FIG. 9, one or more light sources 110 may be provided within the sensor holder 114 in an arrangement that provides light to areas of the skin 112 at or near the injection site of the sensor probe 116. Such an arrangement may be suitable for providing light to modulate the FBR at the injection site and depending on the wavelength, to depths beneath the skin 112 that correspond with tissue regions that include the sensor probe 116.

FIG. 10 is an illustration representing a CGM 108-2 that is similar to the CGM 108-1 of FIG. 9 and further includes a corresponding light delivery structure 122 capable of delivering FBR-modulating light beneath the host's skin 112 during monitoring. The light delivery structure 122 may embody an optical waveguide, such as a fiber optic, that receives light from at least one of the light sources 110 in the CGM 108-2. In certain embodiments, the light source 110 may reside within the sensor holder 114 and the light delivery structure 122 may be mechanically supported by the sensor holder 114. Accordingly, the sensor probe 116 and the light delivery structure 122 may be concurrently inserted beneath the skin 112. The light delivery structure 122 may be suitable for delivering light along portions of the sensor probe 116 in order to modulate the FBR and improve accuracy of the sensor probe 116 over time.

As described herein, principles of the present disclosure provide devices and systems for implementing therapeutic treatments to patients where illumination devices are in communication with local devices and servers. In this regard, treatment protocols may be developed and administered by illumination devices based on diagnostic information specific to a patient in combination with global diagnostic information and efficacy of previously administered treatment protocols. In doing so, the server may analyze and compare patient-specific information with global information to generate tailored treatment protocols. Delivery of the tailored treatment protocols may be provided to the illumination device and/or to a medical provider that may administer the treatment. Since the illumination device is in communication with the server, information may be exchanged while a treatment is being administered to provide real-time monitoring and/or adjustments to treatments. In certain embodiments, such real-time adjustments may allow a patient to receive suitable treatments with reduced treatment time and with reduced side-effects. The server may continuously receive and analyze global treatment information to continuously fine tune the next generated treatment protocols. In this manner, the server and associated synthetic intelligence may be well suited for determining a disease state or other condition of a patient and providing a tailored treatment protocol based on the most up to date information possible. In addition to automated information exchange, systems of the present disclosure may also have manual functions where designated or authorized personnel can update diagnostic information and/or databases or artificial intelligence libraries. While the information is being exchanged and compiled, the server may also be capable of monitoring patient habits and/or compliance with treatment protocols and providing diagnostics related to operation of the illumination devices. In comparison with conventional medications that typically have time delays between initial diagnosis and actual deliver of medication, light treatment protocols according to the present disclosure may be generated and rapidly administered, and associated efficacy may be fed back to the server for refinement of future treatment protocols.

It is contemplated that any of the foregoing aspects, and/or various separate aspects and features as described herein, may be combined for additional advantage. Any of the various embodiments as disclosed herein may be combined with one or more other disclosed embodiments unless indicated to the contrary herein.

Those skilled in the art will recognize improvements and modifications to the preferred embodiments of the present disclosure. All such improvements and modifications are considered within the scope of the concepts disclosed herein and the claims that follow.

What is claimed is:

1. An illumination device for phototherapeutic delivery of light, the illumination device comprising;
a light source;
a communication interface; and
a control system associated with the communication interface, the control system configured to a collect diagnostic information, implement a treatment protocol, and send the diagnostic information and administered light treatment information associated with implementing the treatment protocol to a server via the communication interface.

2. The illumination device of claim 1, further comprising one or more of a sensor and a camera associated with the control system, the one or more of the sensor and the camera being configured to collect at least a portion of the diagnostic information.

3. The illumination device of claim 1, wherein the control system is configured with a pre-configured treatment protocol, and the treatment protocol is modified from the pre-configured treatment protocol based on the diagnostic information.

4. The illumination device of claim 1, wherein the control system is configured to determine the treatment protocol based on the diagnostic information.

5. The illumination device of claim 1, wherein the control system is further configured to receive the treatment protocol from the server based on the diagnostic information.

6. The illumination device of claim 1, wherein the control system is further configured to determine location information associated with the administered light treatment information and send the location information to the server.

7. An illumination device for phototherapeutic delivery of light, the illumination device comprising;
a light source;
a communication interface; and
a control system associated with the communication interface, the control system configured to implement a treatment protocol, determine location information associated with the treatment protocol, and send the location information to a server via the communication interface.

8. The illumination device of claim 7, wherein the location information comprises a global positioning system (GPS) location.

9. The illumination device of claim 7, wherein the control system is further configured to:
collect diagnostic information;
send the diagnostic information to the server; and
receive the treatment protocol from the server and control the light source to implement the treatment protocol.

10. The illumination device of claim 9, wherein the control system is further configured to send the diagnostic information to a local device before sending the diagnostic information to the server.

11. The illumination device of claim 9, wherein the control system is further configured to determine the location information before sending the diagnostic information to the server.

12. The illumination device of claim 9, wherein the control system is further configured to determine the location information after receiving the treatment protocol from the server.

13. The illumination device of claim 9, wherein at least one of the location information, the diagnostic information, and the treatment protocol comprises encrypted data.

14. The illumination device of claim 7, wherein the treatment protocol comprises a pre-configured treatment protocol that is associated with the control system.

15. The illumination device of claim 7, wherein the control system is further configured to receive the treatment protocol from a local device that is in communication with the control system and the server.

16. The illumination device of claim 7, wherein the control system is further configured to send administered light treatment information to the server, the administered light treatment information comprising one or more of a wavelength of light and a dose of light associated with administered light treatment.

17. A system for phototherapeutic delivery of light, the system comprising;
a server; and
a server-side application associated with the server, the server-side application configured to:
receive diagnostic information from at least one of an illumination device and a local device that is in communication with the illumination device;
generate a treatment protocol based on the diagnostic information;
send the treatment protocol to the illumination device; and
receive location information associated with administered light treatment information after the treatment protocol is implemented by the illumination device.

18. The system of claim 17, wherein the server-side application is configured to compile geospatial information based on:
a plurality of treatment protocols generated for a plurality of illumination devices; and
location information associated with administered light treatment information received from the plurality of illumination devices.

19. The system of claim 17, wherein the server-side application is further configured to receive additional user information together with the diagnostic information, the additional user information comprising one or more of a medical history and demographics of a user.

20. The system of claim 17, wherein the server-side application is configured to associate additional user information with the diagnostic information, the additional user information comprising one or more of a medical history and demographics of a user.

21. The system of claim 17, wherein one or more of the diagnostic information, the treatment protocol, and the location information comprises encrypted data.

22. The system of claim 17, wherein the server comprises an artificial intelligence library that is used to generate the treatment protocol based on the diagnostic information.

23. The system of claim 17, wherein the administered light treatment information comprises one or more of a wavelength of light and a dose of light implemented by the illumination device.

24. A system for phototherapeutic delivery of light, the system comprising;
a server; and
a server-side application associated with the server, the server-side application configured to:
receive administered light treatment information from a plurality of illumination devices, the administered light treatment information being associated with location information; and
provide data for compiling geospatial information based on the administered light treatment information and the location information.

25. The system of claim 24, wherein the administered light treatment information comprises one or more of a wavelength of light and a dose of light associated with administered light treatments implemented by the plurality of illumination devices.

26. The system of claim 24, wherein the administered light treatment information is associated with one or more of a user's diagnostic information, medical history, and demographics.

27. The system of claim 26, wherein the server-side application is further configured to receive the administered light treatment information from a local device that is in communication with the plurality of illumination devices.

28. The system of claim 24, wherein the administered light treatment information comprises encrypted data.
